(19) United States
(12) Patent Application Publication
(10) Pub. No.: US 2025/0134942 A1
(43) Pub. Date: May 1, 2025

(54) COMPOSITION FOR IMPROVING MUSCLE STRENGTH, COMPRISING, AS ACTIVE INGREDIENT, AUREOBASIDIUM PULLULANS FERMENTATION PRODUCT WITH INCREASED AMOUNT OF BETAGLUCAN, AND PREPARATION METHOD THEREFOR

(71) Applicant: GLUCAN, INC, Gyeongsangnam-do (KR)

(72) Inventors: Hyung Rae CHO, Busan (KR); Young Suk KIM, Gyeongsangnam-do (KR); Jong Min LIM, Busan (KR); Bon-Hwa KU, Gyeongsangnam-do (KR); Da Mi CHEON, Gyeongsangnam-do (KR)

(73) Assignee: GLUCAN, INC, Gyeongsangnam-do (KR)

(21) Appl. No.: 18/836,170

(22) PCT Filed: Mar. 4, 2022
(86) PCT No.: PCT/KR2022/095048

§ 371 Date: Aug. 6, 2024

(30) Foreign Application Priority Data
Feb. 7, 2022 (KR) 10-2022-0015684

(51) Int. Cl. A61K 36/02; A61K 9/00; A61K 9/16; A61K 9/20; A61K 9/48; A61K 31/716; A61P 21/00; C12N 1/14; C12N 9/26; C12N 9/44; C12P 19/04; C12P 19/16

(52) U.S. Cl. CPC A61K 36/02; A61K 9/0095; A61K 9/1611; A61K 9/1617; A61K 9/2013; A61K 9/2059; A61K 9/4825; A61K 9/4858; A61K 9/4866; A61K 31/716; A61P 21/00; C12N 1/14; C12N 9/2411; C12N 9/2457; C12P 19/04; C12P 19/16; C12Y 302/01041

(57) ABSTRACT

The present disclosure relates to a composition for improving muscle strength, containing, as an active ingredient, an Aureobasidium pullulans fermentation product produced by culturing Aureobasidium pullulans SM2001 (accession number KCCM 10307) in a medium containing rice bran enzyme lysate fermented using an amylase. According to the present disclosure, the amount of beta-glucan can be increased significantly as compared to a conventional Aureobasidium pullulans fermentation product and, thus, the effects on improvement of muscle strength and prevention or treatment of muscle diseases can be increased.

### TECHNICAL FIELD

The present disclosure relates to a composition for improving muscle strength, which contains an Aureobasidium pullulans fermentation product as an active ingredient, and a method for preparing the same. More specifically, it relates to a composition for improving muscle strength, which contains an Aureobasidium pullulans fermentation product with increased beta-glucan content as an active ingredient, and a method for preparing the same.

### BACKGROUND ART

Muscle is the tissue with the highest portion in the human body. Securing of an appropriate amount of muscle mass is essential in order to maintain the functional capacity of the human body and prevent metabolic diseases. Muscle size is controlled by intracellular signaling processes that induce anabolism or catabolismwithin muscles. Muscle protein synthesis is increased when there are more signaling responses that induce the synthesis rather than those that induce the breakdown of muscle protein, and muscle hypertrophy or increase in the number of muscle fibers may occur as a result.

Muscles also increase bone density by promoting calcium inflow. However, as the body ages, redistribution of body fat and body protein occurs due to changes in composition, and at about 50 years of age, the synthesis rate of protein in muscle cells slows down as compared to the rate of degradation, causing rapid muscle degeneration and muscle loss-related diseases. Sarcopenia, which is a type of muscle loss, refers to a state wherein muscle mass is decreased by about 13 to 24%. It shows decrease in protein content, fiber diameter, muscle strength and fatigue resistance. Sarcopenia is caused by various causes such as sepsis, cancer, renal failure, excessive glucocorticoids, denervation, unuse of muscles, aging, etc.

Its causes include gradual decrease in the quantity and quality of skeletal muscle that occurs as aging progresses and weight loss caused by inadequate intake of foods. It is often deeply correlated with aging. Sarcopenia results from imbalance between protein synthesis and breakdown. Sarcopenia can also worsen the quality of life and can easily cause injuries even in everyday lives. In addition, excessive exercise causes muscle fatigue and damage and reduces athletic ability. Muscle injuries include bruises, lacerations, local anemia, strains and severe damage to skeletal muscles. This damage can cause a great deal of pain. If there is serious damage to skeletal muscle, treatments that reduce muscle damage or speed up muscle tissue recovery are likely to accelerate the recovery of muscle strength after exercise. They can also help the recovery of muscle after illness.

Interest in dieting has increased recently. However, rapid weight loss can cause sarcopenia regardless of age, and intense exercise can cause muscle damage. Therefore, researches and efforts are being focused on treatment of muscle loss or increase of muscle mass in response to common muscle loss diseases, and researches are being conducted on treatment of muscle diseases and enhancement of muscle strength.

### DISCLOSURE

### Technical Problem

The present disclosure is directed to providing a food composition for improving muscle strength that can increase muscle mass and minimize muscle loss by increasing the expression of factors related to muscle cell degradation and suppressing muscle cell death while minimizing side effects on the human body.

The present disclosure is also directed to providing a pharmaceutical composition for preventing or treating muscle diseases that can effectively treat various muscle diseases such as amyotrophy and sarcopenia by increasing the expression of factors related to muscle cell degradation and suppressing muscle cell death while minimizing side effects on the human body.

### Technical Solution

According to an aspect of the present disclosure, there is provided a food composition for improving muscle strength, which contains an Aureobasidium pullulans fermentation product produced by culturing Aureobasidium pullulans SM2001 (accession number KCCM 10307) in a medium containing a rice bran enzyme lysate fermented by an amylase.

The rice bran enzyme lysate may be a rice bran enzyme lysate treated with an enzyme mixture in which a pullulanase is added to the amylase.

The Aureobasidium pullulans fermentation product may have a beta-glucan content of 25 to 40 wt %.

The beta-glucan may be a β-1,3/1,6-glucan containing a glucose main chain connected by β-1,3 bonds, wherein at least one of the glucose in the main chain can be linked to another glucose by a β-1,6 bond.

The β-1,3/1,6-glucan can be represented by the following Chemical Formula 1.

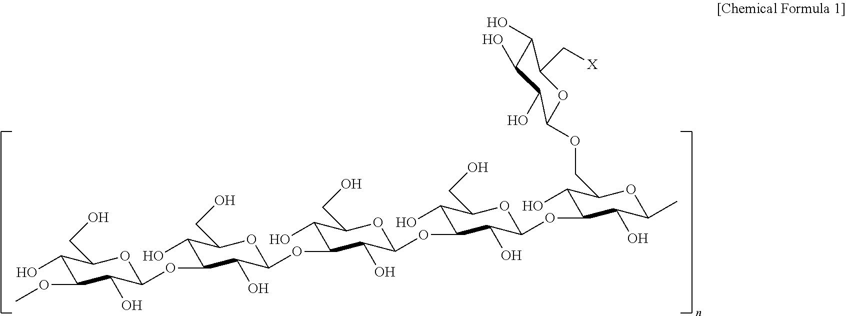

In Chemical Formula 1,
n is n is any integer selected from 1 to 4,000, as the number of repeating units, and
X is any one selected from a lactic acid group, a maleic acid group and a sulfoacetic acid group.

The food composition for improving muscle strength may be for promoting the expression of one or more myogenic factor selected from MyoD, myogenin, MEF2, Myf5 and Myf6.

The food composition for improving muscle strength may be for inhibiting the expression of at least one muscle cell proteolytic factor selected from MuRF1, atrogin-1, FoxO3 and myostatin.

The food composition for improving muscle strength may be for promoting the expression of BCL-2, which is a muscle cell anti-apoptotic factor.

The food composition for improving muscle strength may be for inhibiting the expression of at least one muscle cell pro-apoptotic factor selected from Bax, BAD, Bid, caspase-3 and PARP

According to another aspect of the present disclosure, there is provided a pharmaceutical composition for preventing or treating muscle diseases containing an Aureobasidium pullulans fermentation product produced by culturing Aureobasidium pullulans SM2001 (accession number KCCM 10307) in a medium containing a rice bran enzyme lysate fermented by an amylase.

The muscle disease may be any one selected from muscular atrophy, sarcopenia, atony, muscular dystrophy, myasthenia gravis and amyotrophic lateral sclerosis.

According to another aspect of the present disclosure, there is provided a method for preparing a composition for improving muscle strength, which includes:
(a) a step of preparing a rice bran enzyme lysate by enzymatically treating a mixture of rice bran powder, sugar and water with an amylase;
(b) a step of preparing a medium by mixing water and vitamin C with the rice bran enzyme lysate;
(c) a step of obtaining a culture by culturing Aureobasidium pullulans SM2001 (accession number KCCM 10307) in the medium; and
(d) a step of obtaining an Aureobasidium pullulans fermentation product by removing cells from the culture and concentrating the same.

In the step (a), the enzymatic treatment may be performed by an enzyme mixture in which pullulanase is added to the amylase.

In the step (a), the enzymatic treatment may be performed at 55 to 90° C. for 20 to 100 minutes.

In the step (c), the culturing may be performed by culturing firstly at 20 to 30° C. for 15 to 20 hours and then culturing secondly at 25 to 35° C. for 30 to 40 hours.

### Advantageous Effects

A food composition for improving muscle strength containing an Aureobasidium pullulans fermentation product of the present disclosure can increase muscle mass and minimize muscle loss by increasing beta-glucan content to 25 wt % or higher, thereby increasing the expression of factors related to muscle cell production, reducing the expression of factors related to muscle cell degradation and suppressing muscle cell death while minimizing side effects on the human body.

A pharmaceutical composition for preventing or treating muscle diseases containing an Aureobasidium pullulans fermentation product of the present disclosure can effectively treat various muscle diseases such as amyotrophy, sarcopenia, etc. by increasing beta-glucan content to 25 wt % or higher, thereby increasing the expression of factors related to muscle cell production, reducing the expression of factors related to muscle cell degradation and suppressing muscle cell death while minimizing side effects on the human body.

### BRIEF DESCRIPTION OF DRAWINGS

FIG. 1 shows the result of evaluating cytotoxicity in Experimental Example 1.

FIGS. 2 to 6 show the result of measuring the mRNA expression level of MyoD, myogenin, MEF2, Myf5 and Myf6, which are transcription factors involved in muscle cell generation, in Experimental Example 2.

FIGS. 7 to 10 show the result of measuring the mRNA expression level of MuRF1, atrogin-1, FoxO3 and myostatin, which are muscle cell proteolytic factors, in Experimental Example 2.

FIG. 11 shows the result of measuring the mRNA expression level of BCL-2, which is a muscle cell anti-apoptotic factor, in Experimental Example 3.

FIGS. 12 to 16 show the result of measuring the mRNA expression level of Bax, BAD, BID, caspase-3 and PARP, which are muscle cell pro-apoptotic factors, in Experimental Example 3.

FIG. 17 shows the result of measuring glutathione (GSH) content in Experimental Example 4.

FIG. 18 shows the result of measuring reactive oxygen species (ROS) content in Experimental Example 4.

FIG. 19 shows the result of measuring the protein expression level of MyoD and myogenin, which are transcription factors involved in muscle cell production, in Experimental Example 5.

FIG. 20 and FIG. 21 show the result of measuring the protein expression level of Foxo3a, MurF1 and PI3k, which are signaling pathways factors related to protein synthesis and degradation, in Experimental Example 5.

FIG. 22 shows the result of measuring the expression level of factors related to cell death suppression in Experimental Example 6.

FIG. 23 shows the result of measuring the beta-glucan content of an Aureobasidium pullulans fermentation product of Example 1 in Experimental Example 7.

FIG. 24 shows the result of measuring the beta-glucan content of an Aureobasidium pullulans fermentation product of Comparative Example 1 according to Experimental Example 7.

### BEST MODE

The present disclosure can be modified variously and can have various exemplary embodiments. Hereinafter, specific exemplary embodiments will be described in detail referring to the attached drawings. However, this is not intended to limit the present disclosure to the specific exemplary embodiments and they should be understood to encompass all modifications, equivalents or substitutes included within the scope of the present disclosure. In describing the present disclosure, if it is determined that a specific explanation of a related known technology may obscure the present disclosure, the detailed explanation thereof will be omitted.

A pharmaceutical composition for preventing or treating muscle diseases of the present disclosure contains an Aureobasidium pullulans fermentation product produced by culturing Aureobasidium pullulans SM2001 (accession number KCCM 10307) in a medium containing a rice bran enzyme lysate fermented by an amylase as an active ingredient.

Specifically, the rice bran enzyme lysate may be a rice bran enzyme lysate treated with an enzyme mixture in which pullulanase is added to the amylase.

The Aureobasidium pullulans fermentation product has a beta-glucan content of specifically 25 to 40 wt %, more specifically 30 to 40 wt %, and more specifically 35 to 40 wt %.

The beta-glucan may be a β-1,3/1,6-glucan containing a glucose main chain connected by β-1,3 bonds, wherein at least one of the glucose in the main chain can be linked to another glucose by a β-1,6 bond, and may be represented by Chemical Formula 1.

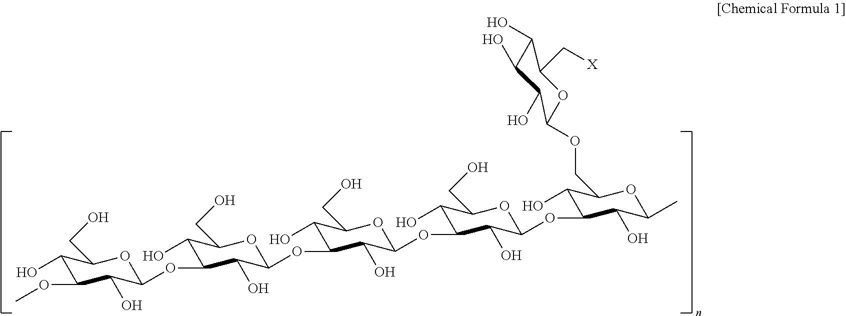

In Chemical Formula 1,
n is n is any integer selected from 1 to 4,000, as the number of repeating units, and
X is any one selected from a lactic acid group, a maleic acid group and a sulfoacetic acid group.

The pharmaceutical composition for preventing or treating muscle diseases may be for increasing the expression of at least one factor related to muscle cell production selected from Myo-D, myogenin, MEF2, Myf5 and Myf6.

Also, the pharmaceutical composition for preventing or treating muscle diseases may be for inhibiting the expression of at least one factor related to muscle cell degradation selected from atrogin-1, MURF-1, FOXO3a and myostatin.

The muscle disease may be any one selected from muscular atrophy, sarcopenia, atony, muscular dystrophy, myasthenia gravis and amyotrophic lateral sclerosis.

In this specification, the term “containing as an active ingredient” means that the Aureobasidium pullulans fermentation product is contained in an amount sufficient to achieve the desired efficacy or activity. In one embodiment of the present disclosure, the composition of the present disclosure contains, for example, 0.001 mg/kg or more, specifically 0.1 mg/kg or more, more specifically 10 mg/kg or more, more specifically 100 mg/kg or more, more specifically 250 mg/kg or more, and most specifically 0.1 g/kg or more. Since the Aureobasidium pullulans fermentation product is a natural product and has no side effect on the human body even when administered in a large amount, those skilled in the art can select the upper limit of the Aureobasidium pullulans fermentation product contained in the composition of the present disclosure within an appropriate range.

In addition to the active ingredient described above, the pharmaceutical composition of the present disclosure can be prepared using a pharmaceutically suitable and physiologically acceptable adjuvant. An excipient, a disintegrant, a sweetener, a binder, a coating agent, an extender, a lubricant, a glidant, a flavorant, etc. can be used as the adjuvant.

Specifically, the pharmaceutical composition may be formulated as a pharmaceutical composition containing one or more pharmaceutically acceptable carrier in addition to the active ingredient described above for administration.

The pharmaceutical composition may be in the form of a granule, a powder, a tablet, a coated tablet, a capsule, a suppository, a liquid, a syrup, a juice, a suspension, an emulsion, a medicinal drop, an injectable liquid, etc. For example, for formulation in the form of a tablet or a capsule, the active ingredient can be combined with an oral, nontoxic, pharmaceutically acceptable inert carrier such as ethanol, glycerol, water, etc. Also, if desired or necessary, a suitable binder, lubricant, disintegrant and colorant or a mixture thereof can be included in the formulation. The suitable binder is are not limited to but includes starch, gelatin, a natural sugar such as glucose or beta-lactose, a corn-based sweetener, a natural and synthetic gum such as acacia, tragacanth or sodium oleate, sodium stearate, magnesium stearate, sodium benzoate, sodium acetate, sodium chloride, etc. The disintegrant is not limited to but includes starch, methyl cellulose, agar, bentonite, xanthan gum, etc.

In a composition formulated as a liquid solution, one or more of saline, sterile water, Ringer's solution, buffered saline, albumin injection solution, dextrose solution, maltodextrin solution, glycerol and ethanol may be used as an acceptable pharmaceutical carrier that is suitable for sterilization and biologically compatible. If necessary, other common additives such as an antioxidant, a buffer, a bacteriostat, etc. can be added. In addition, an excipient, a diluent, a dispersant, a surfactant, a binder, a lubricant, etc. can be additionally added to formulate an injectable formulation such as an aqueous solution, a suspension, an emulsion, etc., a pill, a capsule, a granule or a tablet.

The pharmaceutical composition of the present disclosure can be administered orally or parenterally. For parenteral administration, it can be administered by intravenous infusion, subcutaneous injection, intramuscular injection, abdominal injection, transdermal administration, etc. Specifically, it is administered orally.

An appropriate dosage of the pharmaceutical composition of the present disclosure varies depending on factors such as formulation method, administration method, the age, body weight, sex and pathological condition of a patient, diet, administration time, administration route, excretion rate and response sensitivity. An ordinarily skilled physician can easily determine and prescribe an effective dosage for the desired treatment or prevention. According to a specific exemplary embodiment of the present disclosure, a daily dosage of the pharmaceutical composition of the present disclosure is 0.001-10 g/kg.

The pharmaceutical composition of the present disclosure may be prepared as a unit-dose form using a pharmaceutically acceptable carrier and/or excipient or may be packaged in a multi-dose container according to a method that may be easily carried out by a person having common knowledge in the art to which the present disclosure belongs. The formulation may be in the form of a solution, a suspension or an emulsion in an oily or aqueous medium, or may be in the form of an extract, a powder, a granule, a tablet or a capsule, and may additionally contain a dispersant or a stabilizer.

Furthermore, the present disclosure provides a food composition for improving muscle strength containing an Aureobasidium pullulans fermentation product produced by culturing Aureobasidium pullulans SM2001 (accession number KCCM 10307) in a medium containing a rice bran enzyme lysate fermented by an amylase as an active ingredient.

Since the specific details of the food composition for improving muscle strength of the present disclosure are the same as those of the pharmaceutical composition for preventing or treating muscle diseases described above, the specific details can be referred from the above description.

The food composition according to the present disclosure can be used as a functional food or can be added to various foods. The foods to which the composition of the present disclosure can be added include, for example, beverages, alcoholic beverages, confectionery, diet bars, dairy products, meat, chocolate, pizza, bread, ramen, other noodles, chewing gums, ice creams, vitamin complexes, health supplements, etc.

The food composition of the present disclosure can contain not only the Aureobasidium pullulans fermentation product as an active ingredient, but also ingredients commonly added for food preparation, such as proteins, carbohydrates, fats, nutrients, condiments and flavorants. Examples of the carbohydrate include: common sugars such as monosaccharides, e.g., glucose, fructose, etc.; disaccharides, e.g., sucrose; oligosaccharides; polysaccharides, e.g., dextrin, cyclodextrin, etc., and sugar alcohols such as xylitol, sorbitol, erythritol, etc. As the flavorant, natural flavorants [thaumatin or stevia extract (e.g., rebaudioside A, glycyrrhizin, etc.)] and synthetic flavorants (saccharin, aspartame, etc.) can be used. For example, when the food composition of the present disclosure is prepared as a drink or a beverage, citric acid, high-fructose corn syrup, sugar, glucose, acetic acid, malic acid, fruit juice, various plant extracts can be further added.

The present disclosure provides a health functional food containing a food composition containing the Aureobasidium pullulans fermentation product as an active ingredient. Health functional foods are foods made by adding Aureobasidium pullulans fermentation product to food ingredients such as beverages, teas, spices, chewing gum, and confectionaries, etc., and when ingested, they have specific health effects. But, unlike general pharmaceuticals, they have the advantage of not having side effects that can occur when taking drugs for a long time. The health functional food of the present disclosure obtained in this way is very useful because it can be consumed on a daily basis. The amount of the Aureobasidium pullulans fermentation product added to the health functional food varies depending on the type of the health functional food, but it can be added within a range that does not impair the original taste of the food, usually in the range of 0.01 to 50 wt %, specifically 0.1 to 20 wt %, of the food. And, when the health functional food is in the form of a pill, a granule, a tablet or a capsule, the addition amount of the Aureobasidium pullulans fermentation product may be usually 0.1 to 100 wt %, specifically 0.5 to 80 wt %. In a specific exemplary embodiment, the health functional food of the present disclosure may be in the form of a pill, a tablet, a capsule or a beverage.

Hereinafter, a method for preparing a composition for improving muscle strength of the present disclosure will be described.

First, a mixture of rice bran powder, sugar and water is enzymatically treated with an amylase to prepare a rice bran enzyme lysate (step a).

More specifically, the enzymatic treatment is performed by an enzyme mixture in which pullulanase is added to the amylase.

The enzymatic treatment can be performed at 55 to 90° C. for 20 to 100 minutes.

Next, a medium is prepared by mixing water and vitamin C with the rice bran enzyme lysate (step b).

The medium may be adjusted specifically to pH 5 to pH 6, more specifically to pH 5.2 to pH 5.7.

Afterwards, a culture is obtained by culturing Aureobasidium pullulans (Aureobasidium pullulans SM2001, accession number KCCM 10307) in the medium (step c).

The culturing may be performed specifically by culturing firstly at 20 to 30° C. for 15 to 20 hours and then culturing secondly at 25 to 35° C. for 30 to 40 hours, more specifically by culturing firstly at 23 to 27° C. for 17 to 19 hours and then culturing secondly at 28 to 32° C. for 35 to 37 hours.

Finally, an Aureobasidium pullulans fermentation product is obtained by removing cells from the culture and concentrating the same (step d).

### MODE FOR INVENTION

Hereinafter, specific examples are presented to help understand the present disclosure. However, the following examples only illustrate the present disclosure, and it is obvious to those skilled in the art that various changes and modifications can be made within the scope of the category and technical idea of the present disclosure and that those changes and modifications fall within the scope of the attached claims.

### EXAMPLES

### Preparation Example 1: First Culturing

### (1) Preparation of Rice Bran Enzyme Lysate Using Single Enzyme

After mixing rice bran powder, sugar and water at 1:1.5:5, a rice bran enzyme lysate was prepared by adding 1.5 parts by weight of an amylase as a single enzyme based on 100 parts by weight of the rice bran powder and conducting enzymatic treatment at 70° C. for 1 hour.

### (2) Preparation of First Medium

A first medium was prepared by mixing water with the enzyme lysate prepared using the single enzyme as described above and adding 2% (v/v) of vitamin C based on the total weight.

### Preparation Example 2: Second Culturing

### (1) Preparation of Rice Bran Enzyme Lysate Using Enzyme Mixture

After mixing rice bran powder, sugar and water at 1:1.5:5, a rice bran enzyme lysate was prepared by adding by weight of an enzyme mixture of 1.5 parts amylase and 1.5 parts by weight of pullulanase based on 100 parts by weight of rice bran powder and conducting enzymatic treatment at 70° C. for 1 hour.

### (2) Preparation of the Second Medium

A second medium was prepared by mixing water with the rice bran enzyme lysate prepared using the enzyme mixture as described above and adding 2% (v/v) of vitamin C based on the total weight.

### Example 1: Aureobasidium pullulans Fermentation Product Using First Medium

(1) Culturing of Aureobasidium pullulans Strain

For pre-culturing, Aureobasidium pullulans SM2001 (accession number KCCM 10307) was cultured in a solid medium for a predetermined period of time and inoculated into 100 mL of a medium composition (sugar 1% (v/v), yeast extract 0.2% (v/v), vitamin C 0.2% (v/v)) prepared by sterilizing in a 250-mL flask. Then, a seed culture was prepared by performing shaking culture at 24° C. and 200 rpm for 36 hours.

### (2) Main Culturing

The Aureobasidium pullulans seed culture was sterilized in a 500-mL flask, inoculated to 50 mL of the first medium prepared in Preparation Example 1 at 5% (v/v), and cultured for 18 hours under the condition of 25° C. and 85 rpm with an air flow rate of 1 vvm. Subsequently, culturing was performed for 36 hours under the condition of 30° C. and 170 rpm with an air flow rate of 1 vvm.

### (3) Removal of Cells and Concentration

The culture was centrifuged at 7000×g for 20 minutes, and the supernatant from which cells had been removed was recovered. The recovered supernatant was concentrated under reduced pressure, sterilized at 85° C. for 2 hours, and then freeze-dried to obtain an Aureobasidium pullulans fermentation product (T).

### Example 2: Aureobasidium pullulans Fermentation Product Using Second Medium

An Aureobasidium pullulans fermentation product (TP) was prepared under the same conditions as in Example 1, except that the second medium prepared in Preparation Example 2 was used instead of the first medium prepared in Preparation Example 1 during the main culturing (2).

### Comparative Example 1: Aureobasidium pullulans Fermentation Product Using Conventional Medium

An Aureobasidium pullulans fermentation product (P) was prepared under the same conditions as in Example 1, except that a conventional medium used for fermentation of Aureobasidium pullulans, containing 5 g/L K₂HPO₄, 1 g/L NaCl, 0.2 g/L MgSO₄·7H₂O, 0.6 g/L (NH₄)₂SO₄ and 2.5 g/L yeast extract, was used instead of the first medium prepared in Preparation Example 1 during the main culturing (2).

### EXPERIMENTAL EXAMPLES

### Experimental Example 1: Assessment of Cytotoxicity

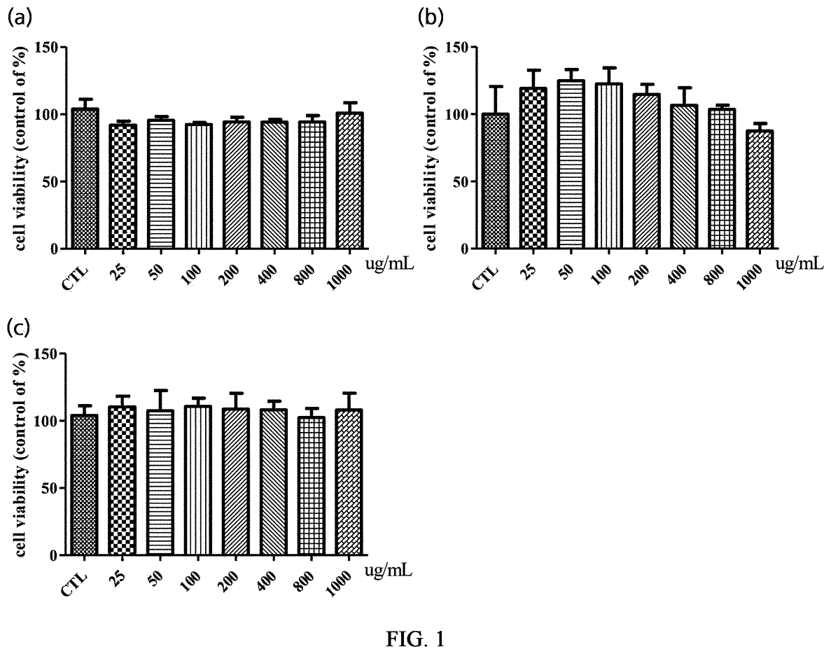

Drug-induced cytotoxicity was investigate using the MTT assay method. 200 μL of C2C12 cells were seeded onto a 96-well plate at a concentration of 1×10⁵ cells/mL and cultured for 24 hours. After treating the C2C12 cells with the Aureobasidium pullulans fermented sample of Example 1 or 2 or Comparative Example 1 at 25, 50, 100, 400, 800 or 1000 μg/mL, followed by incubation for 24 hours, 5 mg/mL (DPBS) of MTT reagent diluted 5 times with a medium was dispensed at 100 μL/well and then reacted in an incubator for 1 hour. After removing the supernatant and dissolving formazan by treating with 100 μL of DMSO, absorbance was measured at 570 nm using a microplate reader (SpectraMaxi3, Molecular Devices, CA, USA). The result is shown in FIG. 1. (a) shows the result for Comparative Example 1 (P), (b) shows the result for the Aureobasidium pullulans fermentation product of Example 1 (T), and (c) shows the result for the Aureobasidium pullulans fermentation product of Example 2 (TP).

There was no effect on the survival rate of the C2C12 cells at all the tested concentrations. Since the cell survival rate at all the concentrations was 90% or higher, it was determined that there was almost no cytotoxicity. Therefore, the highest sample concentration was set to 200 μg/mL in the subsequent experiments.

### Experimental Example 2: Analysis of Expression of Factors Related to Muscle Production and Muscle Cell Proteolysis (qPCR)

C2C12 cells were seeded onto a 6-well culture dish at 3×10⁵ cells/well and cultured for 24 hours and was induced to differentiate for the next 6 days. The cells were treated with the Aureobasidium pullulans fermentation product of Examples 1 and 2 or Comparative Example 1 at a concentration of 100 or 200 g/mL 24 hours before inducing muscle loss. After the muscle loss was induced, the medium was removed. After washing with PBS, RNA was extracted using the RNeasy® mini kit (Aiagen, Hilden, Germany). The extracted RNA was quantified using a spectrophotometer (Nanodrop), and complementary DNA (cDNA) was synthesized from 1 μg of the RNA using the Maxima first strand cDNA synthesis kit for RT-qPCR (Thermo Scientific, Waltham, USA). 19 μL of a mixture containing 10 UL of PCR bio syGREEN blue mix (PCR Biosystems, Pennsylvania, USA) and 2 μL of primers and 1 μL of the cDNA were subjected to 40 cycles of polymerase chain reaction (PCR). The information of the primers used in the polymerase reactions is summarized in Table 1.

TABLE 1

| Target | Primer sequences | |
|---|---|---|
| MyoD | Forward | 5′-GATGGCATGATGGATTACAG-3′ |
| | Reverse | 5′-CTCCACTATGCTGGACAGG-3′ |
| Myogenin | Forward | 5′-AGTACATTGAGCGCCTACAG-3′ |
| | Reverse | 5′-GACGTAAGGGAGTGCAGATT-3′ |
| Atrogin-1 | Forward | 5′-CTGCCTGTGTGCTTACAACT-3′ |
| | Reverse | 5′-TGCTCTCTTCTTGGGTAACA-3′ |
| Myf5 | Forward | 5′-TGAGGGAACAGGTGGAGAAC-3′ |
| | Reverse | 5′-AGCTGGACACGGAGCTTTA-3′ |
| Myf6 | Forward | 5′-ATTCTTGCGGGTGCGGATTT-3′ |
| | Reverse | 5′-ACGTTTGCTCCTCCTTCCTT-3′ |
| MEF2 | Forward | 5′-TCCATCAGCCATTTCAACAA-3′ |
| | Reverse | 5′-GTTACAGAGCCGAGGTGGAG-3′ |
| FoxO3 | Forward | 5′-ACAAACGGCTCACTTTGTCC-3′ |
| | Reverse | 5′-GTGCCGGATGGAGTTCTC-3′ |
| Myostatin | Forward | 5′-CTGTAACCTTCCCAGACCA-3′ |
| | Reverse | 5′-GCAGTCAAGCCCAAAGTCTC-3′ |
| MuRF1 | Forward | 5′-TGCCTACTTGCTCCTTGC-3′ |
| | Reverse | 5′-CACCAGCATGGAGATGCAGT-3′ |

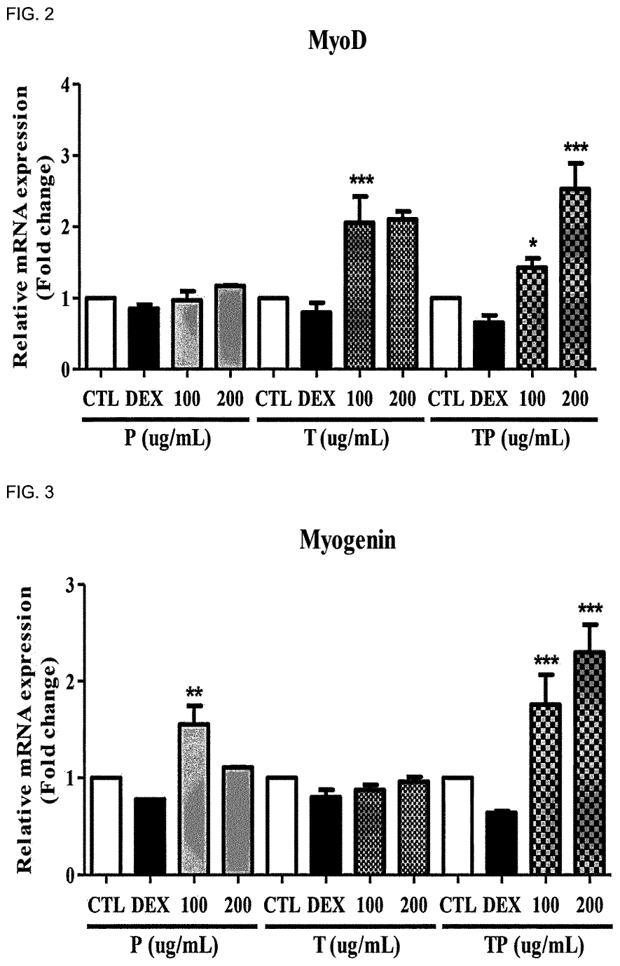

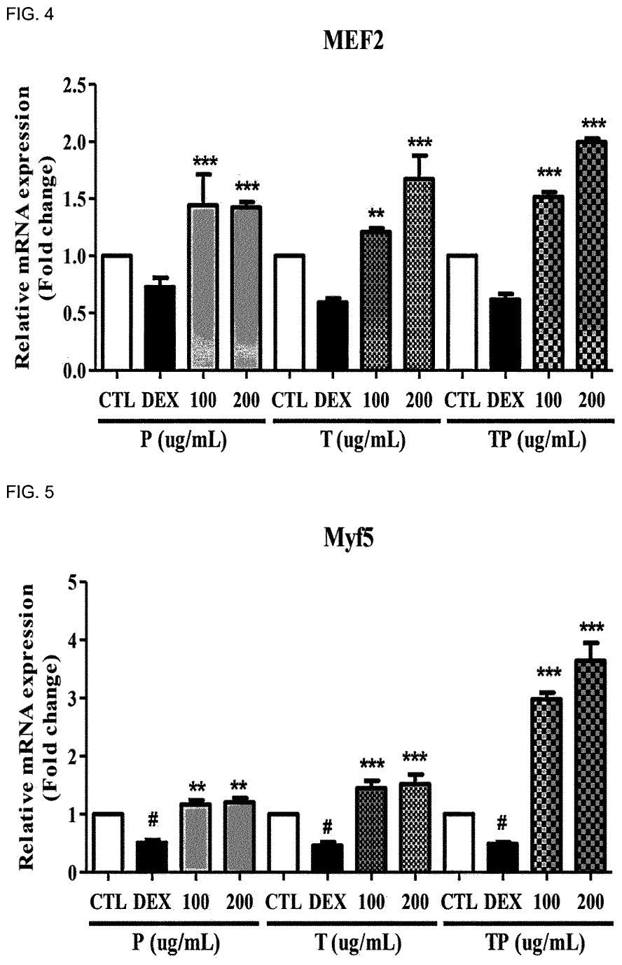

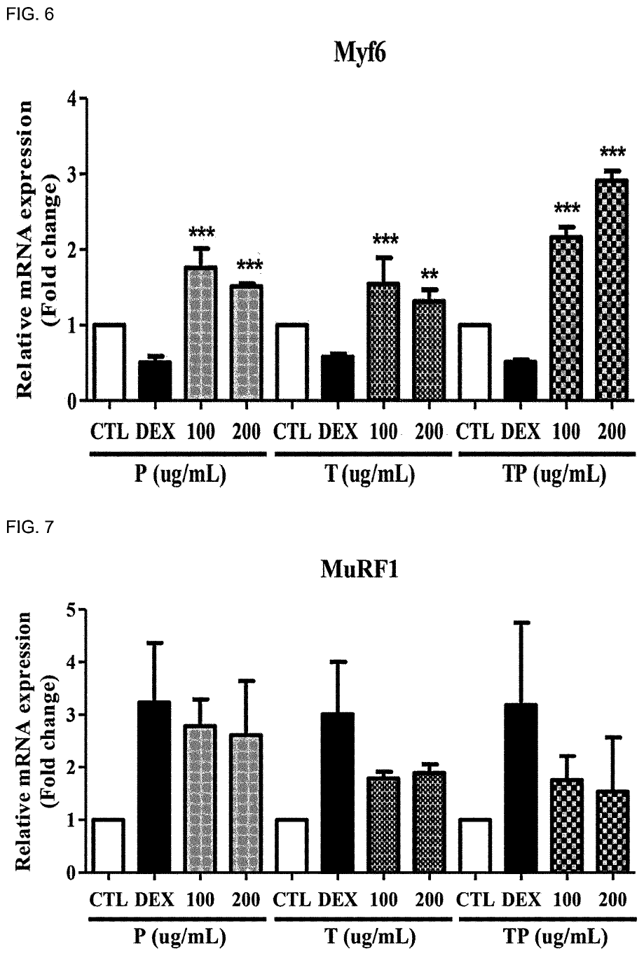

After differentiating C2C12 cells into myotubes, the expression of mRNA was analyzed by treating with the Aureobasidium pullulans fermentation product of Examples 1 and 2 or Comparative Example 1 24 hours before induction of amyotrophy, in order to confirm whether the Aureobasidium pullulans fermentation product of Examples 1 and 2 or Comparative Example 1 has an effect on muscle cell production and proteolytic factors in the cells in a dexamethasone-induced muscle atrophy model. The result of analyzing the mRNA expression level of MyoD, myogenin, MEF2, Myf5 and Myf6, which are transcription factors involved in muscle cell production, is shown in FIGS. 2 to 6, respectively. It was confirmed that the mRNA expression of MyoD, myogenin, MEF2, Myf5 and Myf6 increased significantly in a concentration-dependent manner in the Example 1 treatment group (T) or the Example 2 treatment group (TP) and, in particular, the muscle cell generation transcription factors were expressed at higher levels in the Example 2 treatment group (TP) (#p<0.05, ##p<0.01, ###p<0.001 vs. CTL; * p<0.05, ** p<0.01, *** p<0.001 vs. Dex group).

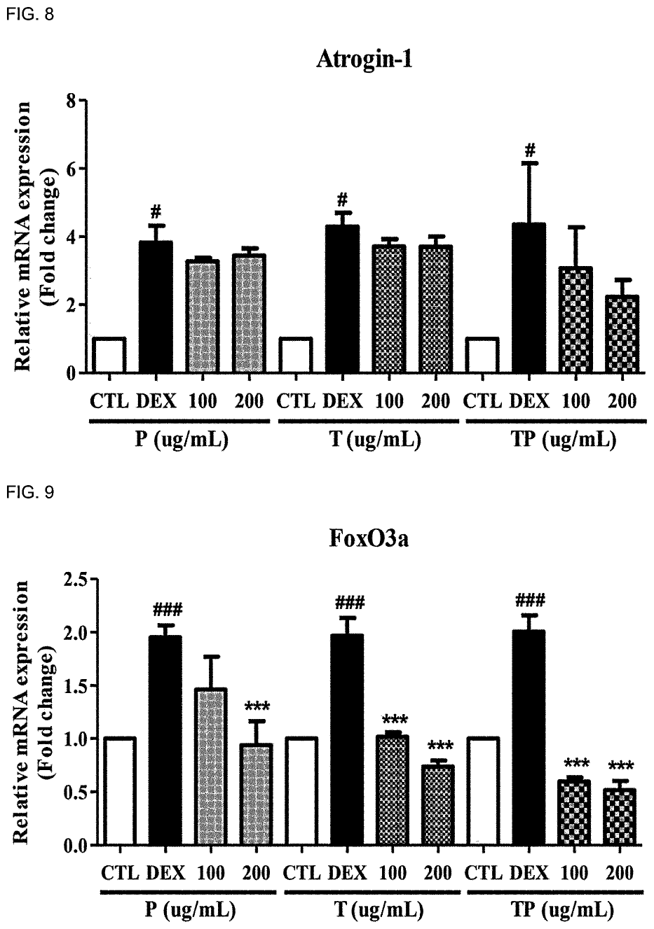

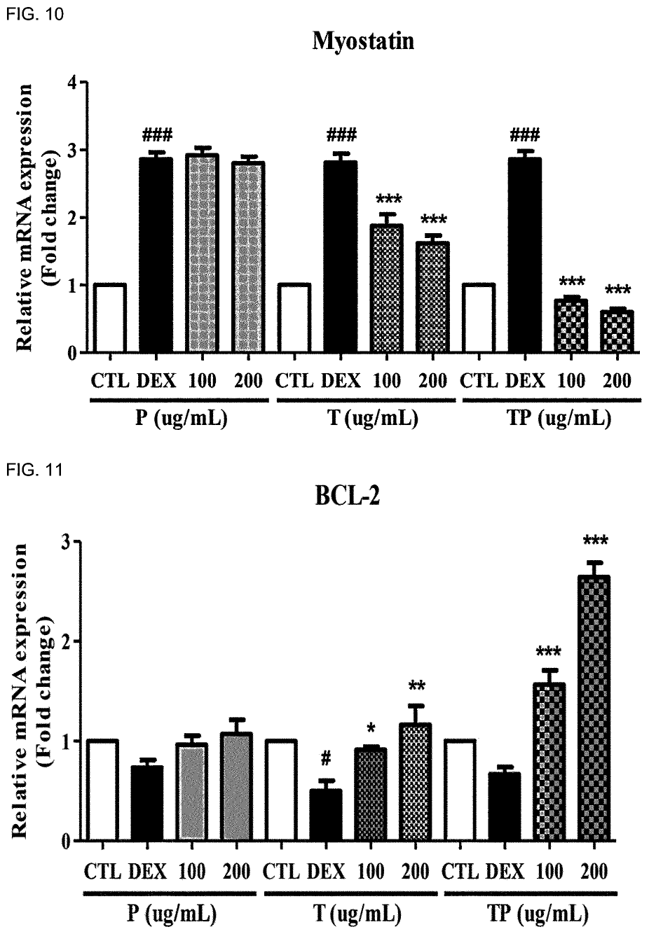

Also, the analysis result of the mRNA expression of MuRF1, atrogin-1, FoxO3 and myostatin, which are muscle cell proteolytic factors, is shown in FIGS. 7 to 10, respectively. It was confirmed that the expression level of MuRF1, atrogin-1, FoxO3 and myostatin decreased significantly in a concentration-dependent manner in the Example 1 treatment group (T) or the Example 2 treatment group (TP) and, in particular, the expression level was lower in the Example 2 treatment group (TP) (#p<0.05, ##p<0.01, ###p<0.001 vs. CTL; * p<0.05, ** p<0.01, *** p<0.001, vs. Dex group).

### Experimental Example 3: Analysis of Expression of Factors Related to Muscle Cell Death (qPCR)

qPCR analysis was performed using the same method as in Experimental Example 2. The information of the primers used in polymerase reaction is summarized in Table 2.

TABLE 2

| Target | Primer sequences | |
|---|---|---|
| BCL-2 | Forward | 5′-GATTTCTCCTGGCTGTCTCT-3′ |
| | Reverse | 5′-TGTGTGTGTGTGTTCTGCTT-3′ |
| Bax | Forward | 5′-CCTTTTTGCTACAGGGTTC-3′ |
| | Reverse | 5′-TCCATATTGCTGTCCAGTTC-3′ |
| BAD | Forward | 5′-CGAAGGATGAGCGATGAGTT-3′ |
| | Reverse | 5′-TAGAGTTCCGGGATGTGGAG-3′ |
| Bid | Forward | 5′-CAGGAAGAAATCATCCACAA-3′ |
| | Reverse | 5′-GCTGCTTCACCTCATCAAG-3′ |
| Caspase-3 | Forward | 5′-TGGTGATGAAGGGGTCATTT-3′ |
| | Reverse | 5′-AGCCTCCACGGGTATCTTCT-3′ |
| PARP | Forward | 5′-ATTCCTAGCCGAAAGGAATGG-3′ |
| | Reverse | 5′-TAGACAGGGGCTTGTCTGCT-3′ |

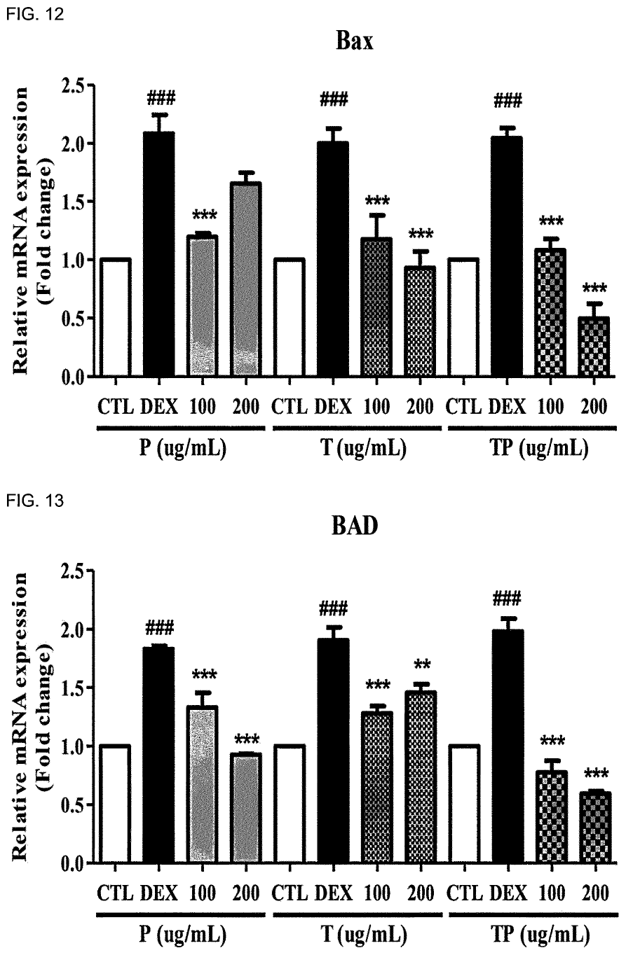

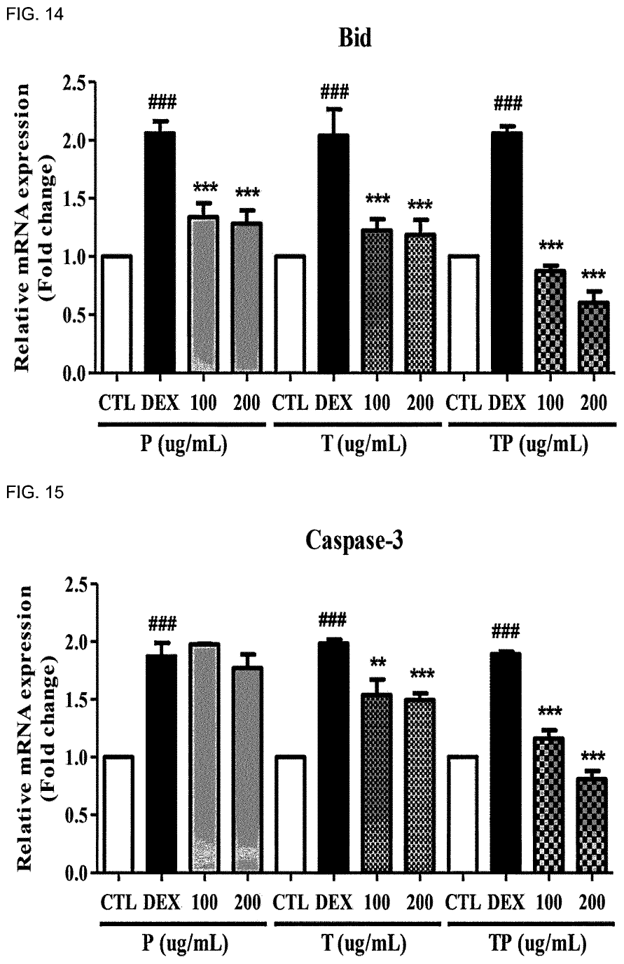

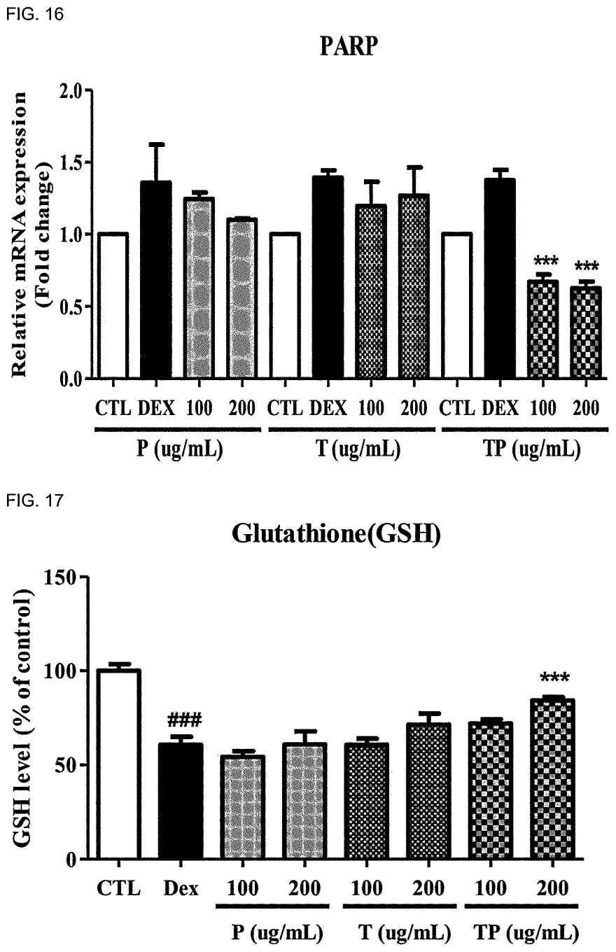

The measurement result of the mRNA expression level of BCL-2, which is a muscle cell anti-apoptotic factor, is shown in FIG. 11. BCL-2 was shown to increase significantly in the group (T) treated with the Aureobasidium pullulans fermentation product of Example 1 and the group (TP) treated with the Aureobasidium pullulans fermentation product of Example 2. In particular, it was measured at a higher level in the group (TP) treated with the Aureobasidium pullulans fermentation product of Example 2. Also, the mRNA expression level of Bax, BAD, Bid, caspase-3 and PARP, which are muscle cell pro-apoptotic factors, was measured and the result is shown in FIGS. 12 to 16, respectively. The expression of the muscle cell pro-apoptotic factors was generally lower in the group (T) treated with the Aureobasidium pullulans fermentation product of Example 1 and the group (TP) treated with the Aureobasidium pullulans fermentation product of Example 2. In particular, it was measured at a lower level in the group (TP) treated with the Aureobasidium pullulans fermentation product of Example 2 (#p<0.05, ##p<0.01, ###p<0.001 vs. CTL; * p<0.05, ** p<0.01, *** p<0.001 vs. Dex group).

### Experimental Example 4: Analysis of Oxidative Stress

The content of the antioxidant glutathione (GSH) in C2C12 muscle cells was measured using a glutathione assay kit. Experiment was conducted by treating with the Aureobasidium pullulans fermentation product of Examples 1 and 2, or Comparative Example 1 at concentrations of 100 and 200 μg/mL, respectively, and the GSH content of the supernatant was measured using a glutathione assay kit (Cayman Co, USA). After adding 150 μL of an analytical cocktail in the kit to 50 μL of the supernatant and shaking with a microshaker in the dark, absorbance was measured at 405 nm using a microreader.

Meanwhile, in order to measure the level of reactive oxygen species (ROS) with oxidative power that attacks living tissue and damages cells in the C2C12 muscle cells, the cells were seeded onto a 24-well plate at 2×10⁵ cells/mL and cultured for 24 hours. When the cells grew to 90%, differentiation was induced by replacing the medium with DMEM containing 2% HS and 1% P/S. The medium was exchanged every 2 days. On day 6 after the differentiation was completed, the Aureobasidium pullulans fermentation product of Examples 1 and 2 or Comparative Example 1 was treated at concentrations of 100 and 200 μg/mL, respectively, and the cells were washed with PBS 24 hours later and then treated with 10 UM dexamethasone to induce muscle atrophy. Then, the cells were washed with PBS, and after dispensing 1 mL of 10 UM DCF-DA to each well, were incubated in an incubator at 37° C. and 5% CO₂ for 30 minutes. 30 minutes later, the cells were wash with PBS. After adding 1 mL of PBS to each well, the level of ROS in the cells was measured by measuring fluorescence at 485/20 nm for excitation and 528/20 nm for emission using a microplate reader.

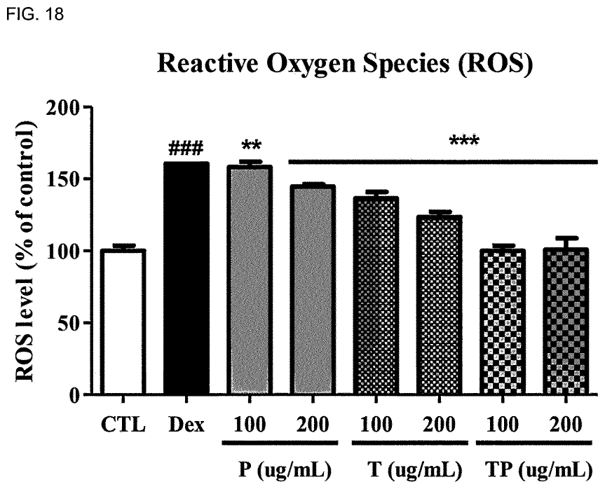

The result of measuring the content of glutathione (GSH) is shown in FIG. 17, and the result of measuring the content of active oxygen species (ROS) is shown in FIG. 18. The glutathione (GSH) content was significantly higher level in the group treated with the Aureobasidium pullulans of Example 2 at 200 μg/mL as compared to the dexamethasone treatment group (Dex). And, the content of active oxygen species (ROS) was significantly lower in the groups treated with the Aureobasidium pullulans fermentation products of Examples 1 and 2 as compared to the dexamethasone treatment group (Dex) (#p<0.05, ##p<0.01, ###p<0.001 vs. CTL; * p<0.05, ** p<0.01, *** p<0.001, vs. Dex group).

### Experimental Example 5: Analysis of Expression of Factors Related to Muscle Production and Muscle Cell Proteolysis (Western Blot)

The expression of proteins related to differentiation and muscle loss in C2C12 cells was measured by western blot. First, after treating C2C12 cells with the Aureobasidium pullulans fermentation product of Examples 1 and 2 or Comparative Example 1 at the nontoxic concentration of 100 or 200 μg/mL, dexamethasone was treated for 24 hours to induce muscle loss. After washing the cells 3 times with 1×PBS, they were lysed with 0.1 mL of a lysis buffer (50 mM HEPES, PH 7.4, 150 mM NaCl, 1% deoxycholate, 1 mM EDTA, 1 mM PMSF, 1 μg/mL aprotinin). Proteins were separated by centrifuging the cell lysate at 12,000 rpm for 20 minutes. The concentration of the separated proteins was quantified with a protein assay solution, and then 30 μg of the proteins were mixed with a 5× sample buffer and separated by 8-15% SDS-PAGE. The separated proteins on the gel were transferred to an NC membrane and the membrane was blocked with 5% BSA for 1 hour at room temperature. After adding primary antibodies related to differentiation, apoptosis, and muscle loss to the membrane and incubating overnight at 4° C., the membrane was washed 3 times with TBS containing 0.05% Tween. After adding anti-igG conjugated HRP antibody to the membrane and incubating at room temperature for 1 hour, the membrane was washed 3 times with TBS (1×TTBS) containing 0.05% Tween and analyzed using a ChemiDoc™ Touch imaging system (BioRad, California, USA) using an ECL solution.

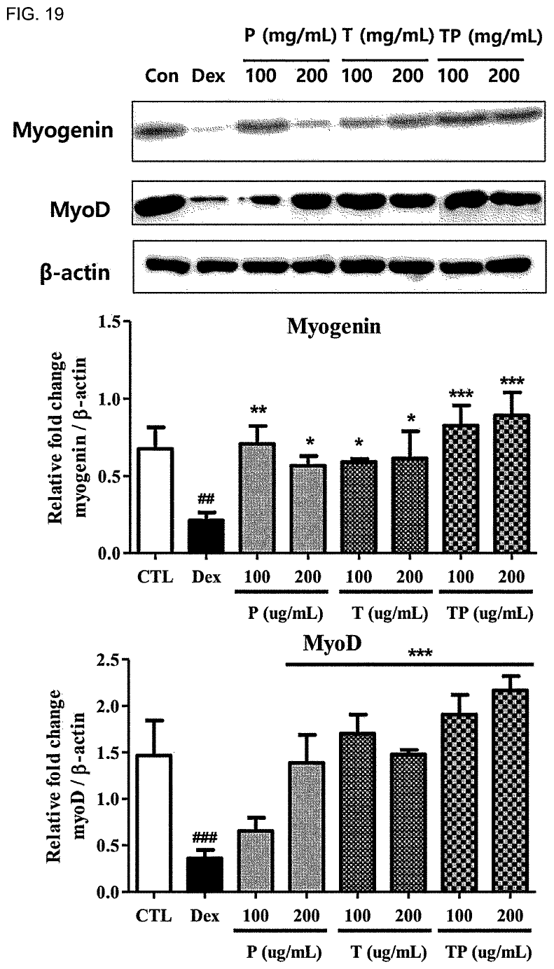

The result of measuring the expression level of MyoD and myogenin proteins, which are transcription factors involved in muscle cell production, is shown in FIG. 19. The expression level of myogenin was the highest in the group treated with the Aureobasidium pullulans fermentation product of Example 2 (TP), and the protein expression level of the MyoD was higher in the groups treated with the Aureobasidium pullulans fermentation product of Example 1 (T) and Example 2 (TP) as compared to the dexamethasone treatment group (Dex).

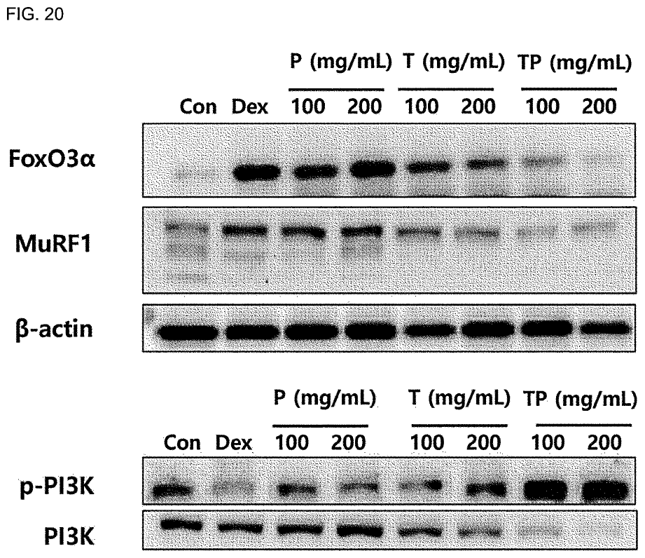

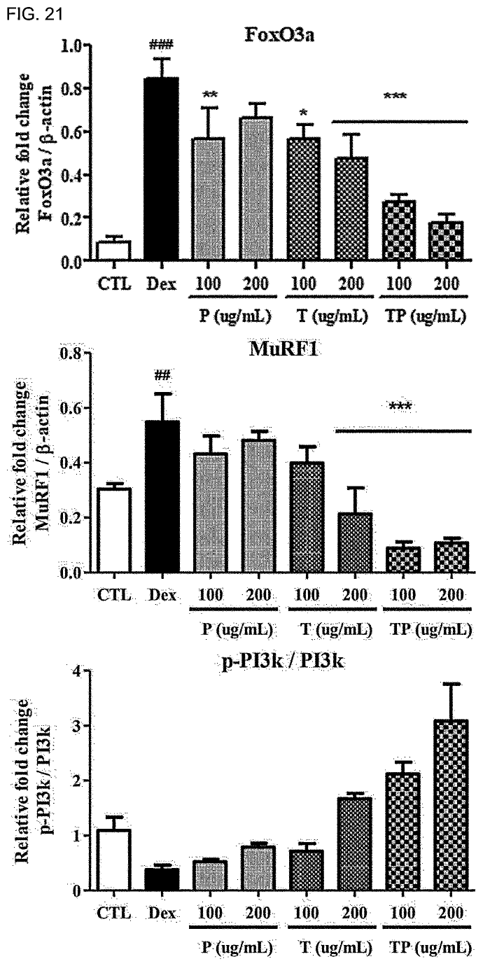

Also, the protein expression level of Foxo3a, MurF1 and PI3k, which are factors involved in signaling pathways related to protein synthesis and degradation, was measured after treating C2C12 myotube cells with the Aureobasidium pullulans fermentation product and dexamethasone for 24 hours in order to investigate the mechanism of action for improvement of muscle strength. The result is shown in FIG. 20 and FIG. 21. It was confirmed that the level of FOXO3α and MURF1 was increased significantly in the dexamethasone treatment group, and the level of Fox03a and MurF1, which are muscle cell proteolytic factors, was decreased significantly in the 200 μg/mL treatment groups of Example 1 (T) and Example 2 (TP). Also, it was confirmed that the degradation of muscle protein was inhibited in the treatment groups of Example 1 (T) and Example 2 (TP) because of increased p-PI3k in the PI3k pathway (#p<0.05, ##p<0.01, ###p<0.001 vs. CTL; * p<0.05, ** p<0.01, *** p<0.001, vs. Dex group).

### Experimental Example 6: Analysis of Expression of Anti-Apoptotic Factors (Western Blot)

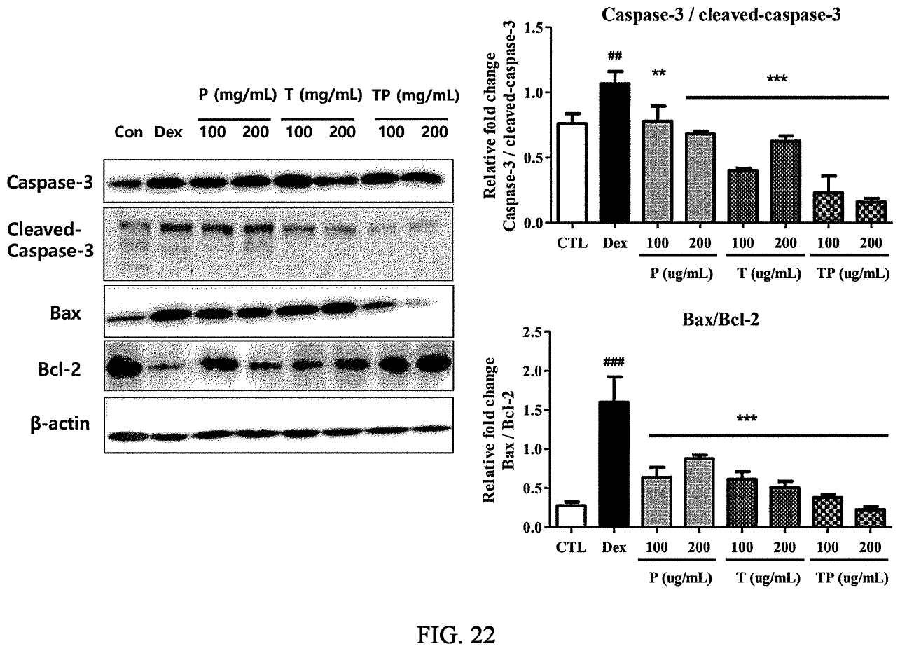

The protein expression level of caspase-3, cleaved-caspase-3, Bax, and Bcl-2 was measured according to the same western blot method as in Experimental Example 5. The result of measuring the caspase-3/cleaved-caspase-3 and Bax/Bcl-2 values is shown in FIG. 22.

It was confirmed that the caspase-3/cleaved-caspase-3 and Bax/Bcl-2 values that induce apoptosis were low in the treatment groups of Example 1 (T) and Example 2 (TP) and, in particular, they were lower in the Example 2 treatment group (TP), suggesting the effect of suppressing muscle cell death.

### Experimental Example 7: Measurement of Beta-Glucan Content

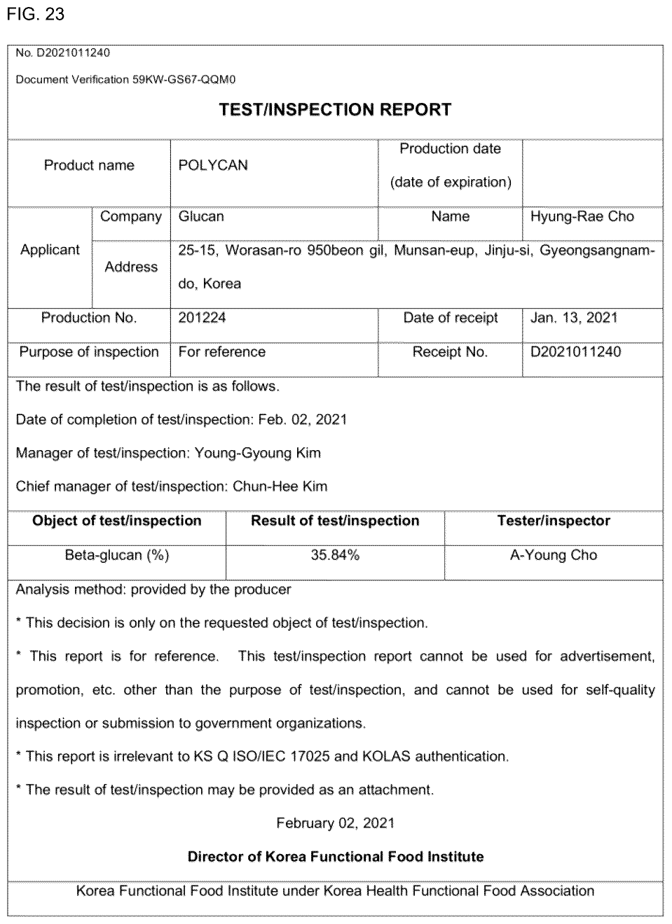

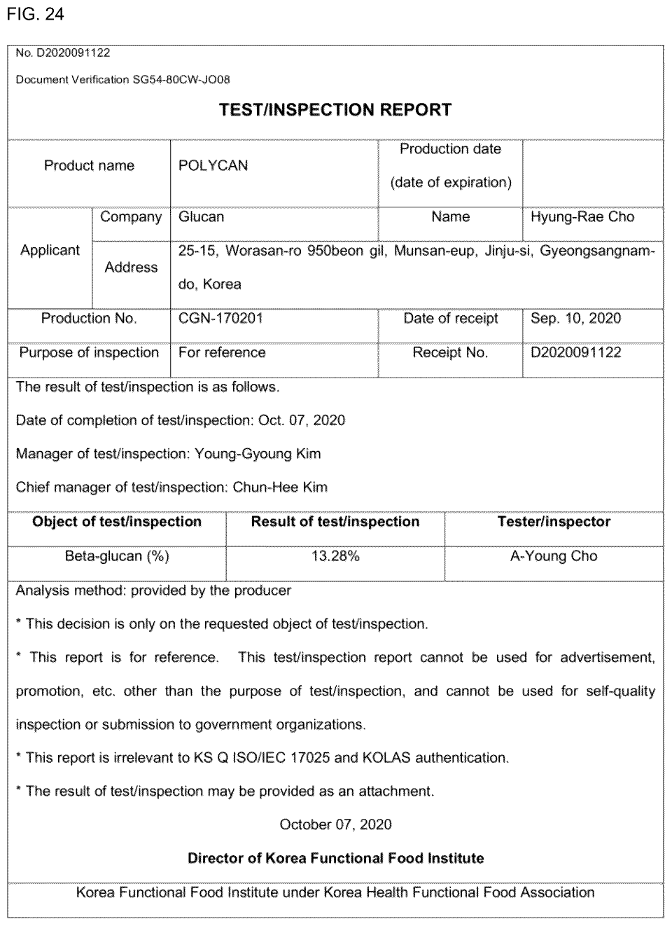

Beta-glucan content was measured for the Aureobasidium pullulans fermentation products of Example 1 and Comparative Example 1. The measurement of beta-glucan content for the Aureobasidium pullulans fermentation product of Example 1 was completed on Feb. 2, 2021, and the test is shown in FIG. 23. The result of measuring beta-glucan content for the conventional Aureobasidium pullulans fermentation product of Comparative Example 1 is shown in FIG. 24.

It was confirmed that, whereas the beta-glucan content was 13.25% for the conventional Aureobasidium pullulans fermentation product of Comparative Example 1, the beta-glucan content was 38.84% for the Aureobasidium pullulans fermentation product of the present disclosure, which was increased by about 3 times as compared to the conventional Aureobasidium pullulans fermentation product.

Hereafter, formulation examples of the composition containing the Aureobasidium pullulans fermentation product of the present disclosure are described. However, they are intended only to specifically explain the present disclosure, not to limit the present disclosure.

### Formulation Example 1. Preparation of Powder

500 mg of Aureobasidium pullulans fermentation product of Example 1 or 2

100 mg of lactose

10 mg of talc

The above ingredients were mixed and filled in an airtight bag to prepare a powder.

### Formulation Example 2. Preparation of Tablet

300 mg of Aureobasidium pullulans fermentation product of Example 1 or 2

100 mg of cornstarch

100 mg of lactose

2 mg of magnesium stearate

After mixing the above ingredients, a tablet was prepared according to a common tablet preparation method.

### Formulation Example 3. Preparation of Capsule

200 mg of Aureobasidium pullulans fermentation product of Example 1 or 2

3 mg of crystalline cellulose

14.8 mg lactose

0.2 mg of magnesium stearate

A capsule was prepared by mixing the above ingredients and filling in a gelatin capsule according to a common capsule preparation method.

### Formulation Example 4. Preparation of Injection

600 mg of Aureobasidium pullulans fermentation product of Example 1 or 2

180 mg mannitol

2974 mg of sterile distilled water for injection

26 mg of Na₂HPO₄·12H₂O

An injection was prepared with the ingredients described above per ampoule according to a common injection preparation method.

### Formulation Example 5. Preparation of Liquid

7.5 g Aureobasidium pullulans fermentation product of Example 1 or 2

10 g of high-fructose corn syrup

5 g of mannitol

Adequate amount of purified water

A liquid was prepared by dissolving the ingredients described above by adding to purified water, adding an appropriate amount of lemon flavor, mixing the same, adding purified water to adjust the total amount to 100 g, filling the liquid in a brown bottle, and then sterilizing the same.

### Formulation Example 6. Preparation of Granule

1,900 mg of Aureobasidium pullulans fermentation product of Example 1 or 2

Adequate amount of vitamin mixture

70 μg of vitamin A acetate

1.0 mg of vitamin E

0.13 mg of vitamin B₁

0.15 mg of vitamin B₂

0.5 mg of vitamin B₆

0.2 μg of vitamin B₁₂

10 mg of vitamin C

10 μg of biotin

1.7 mg of nicotinamide

50 μg of folic acid

0.5 mg of calcium pantothenate

Adequate amount of mineral mixture

1.75 mg of ferrous sulfate

0.82 mg of zinc oxide

25.3 mg of magnesium carbonate

15 mg of potassium monophosphate

55 mg of calcium diphosphate

90 mg of potassium citrate

100 mg of calcium carbonate

24.8 mg of magnesium chloride

The compositional ratios of the vitamin and mineral mixtures described above are presented as specific examples suitable for a granule but may be changed as desired. After mixing the above ingredients, a granule was prepared according to a common granule preparation method for use in the preparation of a health functional food composition.

### Formulation Example 7. Preparation of Functional Drink

1,900 mg of Aureobasidium pullulans fermentation product of Example 1 or 2

1,000 mg of citric acid

100 g of oligosaccharide

2 g of plum concentrate

1 g of taurine

Purified water added to a total volume of 900 mL

According to a common health drink preparation method, the above ingredients were mixed and then heated at 85° C. for about 1 hour under stirring. The resulting solution was filtered into a sterilized 2-L container, sealed, sterilized and then refrigerated for use in the preparation of a functional drink composition.

Although the above compositional ratio is presented as a specific example, it can be changed as desired according to regional and ethnic preferences.

Although the exemplary embodiments of the present disclosure have been described above, those skilled in the art can add, change or delete elements without departing from the spirit of the present disclosure as set forth in the patent claims. The present disclosure may be modified and changed in various ways, which will also be included within the scope of the present disclosure.

### INDUSTRIAL APPLICABILITY

A food composition for improving muscle strength containing an Aureobasidium pullulans fermentation product of the present disclosure can increase muscle mass and minimize muscle loss by increasing beta-glucan content to 25 wt % or higher, thereby increasing the expression of factors related to muscle cell production, reducing the expression of factors related to muscle cell degradation and suppressing muscle cell death while minimizing side effects on the human body.

A pharmaceutical composition for preventing or treating muscle diseases containing an Aureobasidium pullulans fermentation product of the present disclosure can effectively treat various muscle diseases such as amyotrophy, sarcopenia, etc. by increasing beta-glucan content to 25 wt % or higher, thereby increasing the expression of factors related to muscle cell production, reducing the expression of factors related to muscle cell degradation and suppressing muscle cell death while minimizing side effects on the human body.

1. A food composition for improving muscle strength, comprising an Aureobasidium pullulans fermentation product produced by culturing Aureobasidium pullulans SM2001 (accession number KCCM 10307) in a medium comprising a rice bran enzyme lysate fermented by an amylase as an active ingredient.

2. The food composition for improving muscle strength according to claim 1, wherein the rice bran enzyme lysate is a rice bran enzyme lysate treated with an enzyme mixture in which pullulanase is added to the amylase.

3. The food composition for improving muscle strength according to claim 1, wherein the Aureobasidium pullulans fermentation product has a beta-glucan content of 25 to 40 wt %.

4. The food composition for improving muscle strength according to claim 3, wherein the beta-glucan is a β-1,3/1,6-glucan comprising a glucose main chain connected by β-1,3 bonds, wherein at least one of the glucose in the main chain is linked to another glucose by a β-1,6 bond.

5. The food composition for improving muscle strength according to claim 4, wherein the β-1,3/1,6-glucan is represented by the following Chemical Formula 1: 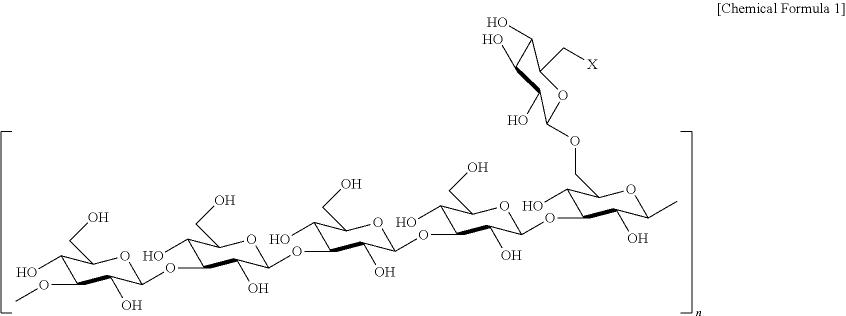
wherein
n is n is any integer selected from 1 to 4,000, as the number of repeating units, and
X is any one selected from a lactic acid group, a maleic acid group and a sulfoacetic acid group.

6. The food composition for improving muscle strength according to claim 1, which is for promoting the expression of one or more myogenic factor selected from MyoD, myogenin, MEF2, Myf5 and Myf6.

7. The food composition for improving muscle strength according to claim 1, which is for inhibiting the expression of at least one muscle cell proteolytic factor selected from MuRF1, atrogin-1, FoxO3 and myostatin.

8. The food composition for improving muscle strength according to claim 1, which is for promoting the expression of BCL-2, which is a muscle cell anti-apoptotic factor.

9. The food composition for improving muscle strength according to claim 1, which is for inhibiting the expression of at least one muscle cell pro-apoptotic factor selected from Bax, BAD, Bid, caspase-3 and PARP.

10. A pharmaceutical composition for preventing or treating muscle diseases, comprising an Aureobasidium pullulans fermentation product produced by culturing Aureobasidium pullulans SM2001 (accession number KCCM 10307) in a medium comprising a rice bran enzyme lysate fermented by an amylase as an active ingredient.

11. The pharmaceutical composition for preventing or treating a muscle disease according to claim 10, wherein the muscle disease is any one selected from muscular atrophy, sarcopenia, atony, muscular dystrophy, myasthenia gravis and amyotrophic lateral sclerosis.

12. A method for preparing a composition for improving muscle strength, comprising:
(a) a step of preparing a rice bran enzyme lysate by enzymatically treating a mixture of rice bran powder, sugar and water with an amylase;
(b) a step of preparing a medium by mixing water and vitamin C with the rice bran enzyme lysate;
(c) a step of obtaining a culture by culturing Aureobasidium pullulans SM2001 (accession number KCCM 10307) in the medium; and
(d) a step of obtaining an Aureobasidium pullulans fermentation product by removing cells from the culture and concentrating the same.

13. The method for preparing a composition for improving muscle strength according to claim 12, wherein, in the step (a), the enzymatic treatment is performed by an enzyme mixture in which pullulanase is added to the amylase.

14. The method for preparing a composition for improving muscle strength according to claim 12, wherein, in the step (a), the enzymatic treatment is performed at 55 to 90° C. for 20 to 100 minutes.

15. The method for preparing a composition for improving muscle strength according to claim 12, wherein, in the step (c), the culturing is performed by culturing firstly at 20 to 30° C. for 15 to 20 hours and then culturing secondly at 25 to 35° C. for 30 to 40 hours.


SEQUENCE LISTING (30 sequences)

SEQ ID NO: 1 - RNA, 20 nt, Artificial Sequence
  MyoD Forward
gatggcatgatggattacag

SEQ ID NO: 2 - RNA, 19 nt, Artificial Sequence
  MyoD Reverse
ctccactatgctggacagg

SEQ ID NO: 3 - RNA, 20 nt, Artificial Sequence
  Myogenin Forward
agtacattgagcgcctacag

SEQ ID NO: 4 - RNA, 20 nt, Artificial Sequence
  Myogenin Reverse
gacgtaagggagtgcagatt

SEQ ID NO: 5 - RNA, 20 nt, Artificial Sequence
  Atrogin1 Forward
ctgcctgtgtgcttacaact

SEQ ID NO: 6 - RNA, 20 nt, Artificial Sequence
  Atrogin1 Reverse
tgctctcttcttgggtaaca

SEQ ID NO: 7 - RNA, 20 nt, Artificial Sequence
  Myf5 Forward
tgagggaacaggtggagaac

SEQ ID NO: 8 - RNA, 20 nt, Artificial Sequence
  Myf5 Reverse
agctggacacggagctttta

SEQ ID NO: 9 - RNA, 20 nt, Artificial Sequence
  Myf6 Forward
attcttgcgggtgcggattt

SEQ ID NO: 10 - RNA, 20 nt, Artificial Sequence
  Myf6 Reverse
acgtttgctcctccttcctt

SEQ ID NO: 11 - RNA, 20 nt, Artificial Sequence
  MEF2 Forward
tccatcagccatttcaacaa

SEQ ID NO: 12 - RNA, 20 nt, Artificial Sequence
  MEF2 Reverse
gttacagagccgaggtggag

SEQ ID NO: 13 - RNA, 20 nt, Artificial Sequence
  FoxO3 Forward
acaaacggctcactttgtcc

SEQ ID NO: 14 - RNA, 19 nt, Artificial Sequence
  FoxO3 Reverse
gtgccggatggagttcttc

SEQ ID NO: 15 - RNA, 20 nt, Artificial Sequence
  Myostatin Forward
ctgtaaccttcccaggacca

SEQ ID NO: 16 - RNA, 20 nt, Artificial Sequence
  Myostatin Reverse
gcagtcaagcccaaagtctc

SEQ ID NO: 17 - RNA, 20 nt, Artificial Sequence
  MuRF1 Forward
tgcctacttgctccttgtgc

SEQ ID NO: 18 - RNA, 20 nt, Artificial Sequence
  MuRF1 Reverse
caccagcatggagatgcagt

SEQ ID NO: 19 - RNA, 20 nt, Artificial Sequence
  BCL2 Forward
gatttctcctggctgtctct

SEQ ID NO: 20 - RNA, 20 nt, Artificial Sequence
  BCL2 Reverse
tgtgtgtgtgtgttctgctt

SEQ ID NO: 21 - RNA, 19 nt, Artificial Sequence
  Bax Forward
cctttttgctacagggttc

SEQ ID NO: 22 - RNA, 20 nt, Artificial Sequence
  Bax Reverse
tccatattgctgtccagttc

SEQ ID NO: 23 - RNA, 20 nt, Artificial Sequence
  BAD Forward
cgaaggatgagcgatgagtt

SEQ ID NO: 24 - RNA, 20 nt, Artificial Sequence
  BAD Reverse
tagagttccgggatgtggag

SEQ ID NO: 25 - RNA, 20 nt, Artificial Sequence
  Bid Forward
caggaagaaatcatccacaa

SEQ ID NO: 26 - RNA, 19 nt, Artificial Sequence
  Bid Reverse
gctgcttcacctcatcaag

SEQ ID NO: 27 - RNA, 20 nt, Artificial Sequence
  Caspase3 Forward
tggtgatgaaggggtcattt

SEQ ID NO: 28 - RNA, 20 nt, Artificial Sequence
  Caspase3 Reverse
agcctccaccggtatcttct

SEQ ID NO: 29 - RNA, 21 nt, Artificial Sequence
  PARP Forward
attcctagccgaaaggaatgg

SEQ ID NO: 30 - RNA, 20 nt, Artificial Sequence
  PARP Reverse
tagacaggggcttgtctgct